(19) United States
(12) Patent Application Publication
(10) Pub. No.: US 2022/0023464 A1
(43) Pub. Date: Jan. 27, 2022

(54) ULTRAVIOLET IRRADIATOR

(71) Applicant: JINS HOLDINGS Inc., Maebashi-shi (JP)

(72) Inventors: Daisuke KANEDA, Maebashi-shi (JP); Azumi KAMATA, Maebashi-shi (JP)

(21) Appl. No.: 17/366,034

(22) Filed: Jul. 2, 2021

(51) Int. Cl. A61L 2/10; A45C 11/04; A45C 15/00; G01J 1/42

(52) U.S. Cl. CPC A61L 2/10; A45C 11/04; A61L 2202/14; G01J 1/42; A61L 2202/11; A45C 15/00

(57) ABSTRACT

An ultraviolet irradiator irradiates eyewear with an ultraviolet ray. The ultraviolet irradiator includes a base stored in an eyewear case that accommodates the eyewear, an irradiation element fixed to the base and irradiating the eyewear accommodated in the eyewear case with the ultraviolet ray, and a switch for turning on the irradiation element. The base is attachable to and detachable from the eyewear case.

### CROSS-REFERENCE TO RELATED APPLICATION

This application is based upon and claims the benefit of priority from Japanese Patent Application No. P2020-125624, filed on Jul. 22, 2020, the entire contents of which are incorporated herein by reference.

### BACKGROUND

### Field

The present disclosure relates to an ultraviolet irradiator that irradiates eyewear with an ultraviolet ray.

### Description of the Related Art

Examples of an ultraviolet irradiator that irradiates eyewear with an ultraviolet ray include a spectacle case disclosed in Japanese Unexamined Patent Publication No. H10-85022. The spectacle case includes an upper case, a lower case pivotally connected to the upper case via a hinge, and a pair of lamps provided on an inner surface of the upper case. The pair of lamps are fixed to a recessed portion inside the upper case to face each of lenses of the spectacle, and irradiate each of the pair of lenses with the ultraviolet ray.

A charger is fixed to the inner surface of the upper case, and a lead wire having a plug attached to a tip extends from the charger. A protruding portion that protrudes inward is formed in a center inside a front edge portion of the upper case, and a hook for engaging with the lower case is provided in the protruding portion. The protruding portion that protrudes inward is formed in the center inside the front edge portion of the lower case, and a hook hole for engaging with the hook of the upper case is formed in the protruding portion.

Since the hook of the upper case engages with the hook hole of the lower case, a state where the upper case is closed by the lower case is maintained. A case switch is provided to protrude upward on a back side of the hook hole in the protruding portion of the lower case. The case switch is a push button type switch, and when the upper case is closed, the protruding portion of the upper case comes into contact with and pushes the case switch.

A manual switch is disposed in a left corner on a front surface of the lower case. The manual switch is a switch for manually operating the above-described pair of lamps to be turned on and off. When the spectacle is not put in the case, or when anti-fog performance of the spectacle is sufficient, the manual switch is turned off.

The spectacle case includes an electric circuit for supplying electric power to the above-described pair of lamps. In the electric circuit, the above-described case switch and the manual switch are disposed in series. Therefore, when the upper case is closed by the lower case, the case switch is turned on and the manual switch is turned on, the pair of lamps are turned on to irradiate each of a pair of lenses of the spectacles with the ultraviolet ray.

In the above-described spectacle case, the pair of lamps are fixed to the upper case, and the electric circuit for turning on the pair of lamps is incorporated in the spectacle case. The above-described spectacle case is a special spectacle case in which the pair of lamps are fixed and the electric circuit is incorporated, and an eyewear case for accommodating the spectacles and ultraviolet irradiation means are integrated with each other. Therefore, in the above-described spectacle case, even in a case of holding a ready-made eyewear case, in a case where a user wants to use an ultraviolet irradiation function, the user has to purchase the spectacle case as a whole, and the ready-made eyewear case is no longer necessary. Accordingly, the spectacle case in the related art is not economical. That is, a current situation is that the irradiation function cannot be diverted to the ready-made eyewear case.

In addition, in the above-described spectacle case, the lamp is provided in the upper case. The case switch is provided in the protruding portion in the center inside the front edge portion of the lower case. The manual switch is provided on the front surface of the lower case. Furthermore, the electric circuit for turning on and off the switches is incorporated therein. Therefore, electrical wiring has to be laid in a limited region of the upper case and the lower case. Consequently, there is a problem in that a configuration may be complicated.

### SUMMARY

An ultraviolet irradiator irradiates an eyewear with an ultraviolet ray. The ultraviolet irradiator includes a base configured to be stored in an eyewear case that accommodates the eyewear, an irradiator fixed to the base and irradiating the eyewear accommodated in the eyewear case with the ultraviolet ray, and a switch configured to turn on the irradiator. The base is attachable to the eyewear case and detachable from the eyewear case.

### BRIEF DESCRIPTION OF THE DRAWINGS

FIG. 1 is a perspective view illustrating an eyewear case as an example to which an ultraviolet irradiator according to an example is applied.

FIG. 2 is a perspective view illustrating an eyewear as an example mounted on the ultraviolet irradiator according to the example in a state where the eyewear case in FIG. 1 is open.

FIG. 3 is a cross-sectional view schematically illustrating a base, an irradiation element, a cushion portion, and the eyewear of the ultraviolet irradiator in FIG. 2.

FIG. 4 is a perspective view illustrating the ultraviolet irradiator and the eyewear case in an open state in FIG. 2.

FIG. 5 is a perspective view illustrating the ultraviolet irradiator in FIG. 2.

FIG. 6 is a perspective view when the ultraviolet irradiator in FIG. 5 is viewed in a direction different from that in FIG. 5.

FIG. 7 is a block diagram illustrating a sensor, a controller, the irradiation element, and notification means of the ultraviolet irradiator in FIG. 2.

FIG. 8 is a perspective view illustrating an ultraviolet irradiator according to a modification example.

### DETAILED DESCRIPTION

In the following description, with reference to the drawings, the same reference numbers are assigned to the same components or to similar components having the same function, and overlapping description is omitted.

Hereinafter, an example of an ultraviolet irradiator according to the present disclosure will be described with reference to the drawings. In the description of the drawings, the same reference numerals will be assigned to the same or equivalent elements, and repeated description will be appropriately omitted. In addition, the drawings may be partially simplified or exaggerated to facilitate understanding, and dimensional ratios are not limited to those illustrated in the drawings.

In the present disclosure, an “eyewear” refers to an instrument worn to cover an eye of a wearer. The “eyewear” includes a spectacle for correcting refractive errors of the eye such as myopia, hyperopia, or astigmatism, sunglasses for decorating a periphery of the eye, and goggles for protecting the eye. In the present example, the eyewear is irradiated with an ultraviolet ray. The “ultraviolet ray” refers to light having a wavelength of 10 nm or longer and 420 nm or shorter. A lower limit of a wavelength band of the “ultraviolet ray” may be 100 nm, 150 nm, 200 nm, 250 nm, or 300 nm. In addition, an upper limit of the wavelength band of the “ultraviolet ray” may be 400 nm, 380 nm, or 350 nm. The wavelength band of the “ultraviolet ray” may include a portion of the wavelength band of visible light. Various purposes can be described as purposes of irradiating the eyewear with the ultraviolet ray. As an example, one of the purposes of irradiating the eyewear with the ultraviolet ray is sterilization. However, for example, the purpose may be anti-fog, and is not particularly limited.

The eyewear is accommodated in the eyewear case. In the present disclosure, the “eyewear case” is a container in which the eyewear can be placed. For example, the “eyewear case” may be a box-shaped container or a bag-shaped container. In this way, the ultraviolet irradiator according to the present example is applicable to various eyewear cases.

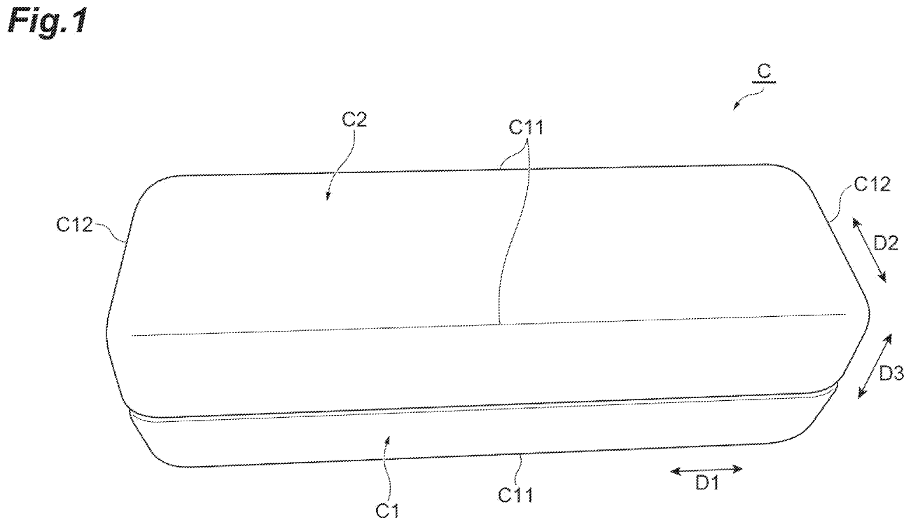

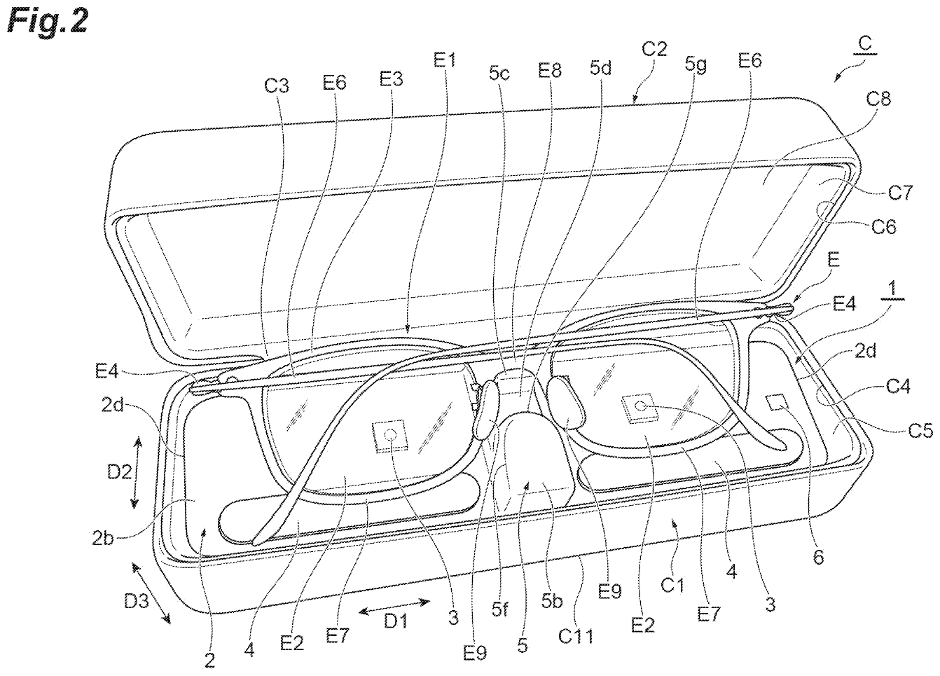

FIG. 1 is a perspective view illustrating an appearance of an eyewear case C as an example to which an ultraviolet irradiator 1 according to the present example is applied. FIG. 2 is a perspective view illustrating the eyewear case C, the ultraviolet irradiator 1, and eyewear E in an open state. As illustrated in FIGS. 1 and 2, the eyewear E is accommodated inside the eyewear case C together with the ultraviolet irradiator 1. The eyewear case C as an example has a rectangular parallelepiped shape having rounded corners.

The eyewear case C has a plurality of long sides C11 extending in a first direction D1 and a plurality of short sides C12 extending in a second direction D2 intersecting the first direction D1, and has a thickness in a third direction D3 intersecting both the first direction D1 and the second direction D2. The eyewear case C includes a mounting portion C1 on which the ultraviolet irradiator 1 and the eyewear E are mounted, and a lid portion C2 that covers the ultraviolet irradiator 1 and the eyewear E which are mounted on the mounting portion C1.

The mounting portion C1 has an opening C4 facing upward. As an example, the opening C4 has a rectangular shape having rounded corners. The lid portion C2 is a portion that functions as a lid for opening and closing the opening C4 of the mounting portion C1. The lid portion C2 is openable and closable with respect to the mounting portion C1 via a hinge portion C3 extending in the first direction D1 on one side (back side) in the second direction D2. That is, the lid portion C2 opens and closes the opening C4 of the mounting portion C1 on which the ultraviolet irradiator 1 and the eyewear E are mounted via the hinge portion C3.

For example, the mounting portion C1 has a box shape having an opening C4, an inner surface C5 extending downward from the opening C4, and a bottom surface on which the ultraviolet irradiator 1 is mounted. For example, the lid portion C2 has a box shape the same as that of the mounting portion C1, and has an opening C6, an inner surface C7, and a bottom surface C8. The hinge portion C3 is provided between an end portion in the second direction D2 in the opening C4 of the mounting portion C1 and an end portion in the second direction D2 in the opening C6 of the lid portion C2.

Hitherto, a configuration of the eyewear case C as an example has been described. However, the configuration of the eyewear case is not limited to the configuration of the above-described eyewear case C. Furthermore, the ultraviolet irradiator 1 and the eyewear E according to the present example can be accommodated in various eyewear cases in addition to the eyewear case C.

The eyewear E as an example includes a frame E1 and a pair of lenses E2 fixed to the frame E1. The frame E1 includes a front E3, a pair of hinges E4 provided in each of both end sides of the front E3, and a pair of temples E6 extending from each of the hinges E4 in a direction intersecting the front E3. The front E3 has a pair of rims E7 surrounding the lens E2, a bridge E8 connecting the pair of rims E7 to each other, and a pair of pads E9 located below the bridge E8. As in the eyewear case, the eyewear is not limited to the configuration of the above-described eyewear E. In the present example, the eyewear E accommodated in the eyewear case C will be described.

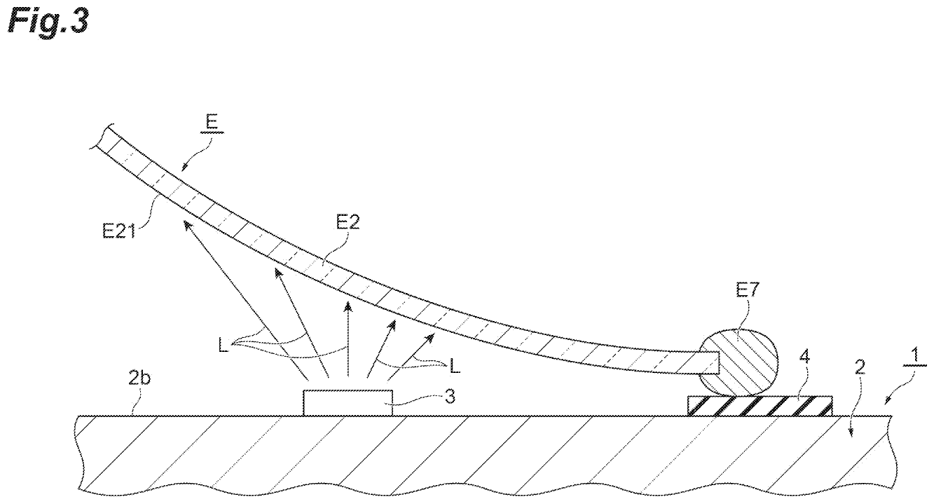

As illustrated in FIGS. 2 and 3, the ultraviolet irradiator 1 according to the present example irradiates the eyewear E accommodated in the eyewear case C with an ultraviolet ray L. The ultraviolet irradiator 1 is attachable to and detachable from the eyewear case C. The description of “attachable and detachable” means a state where the ultraviolet irradiator 1 can be mounted and removed. For example, the description of “attachable and detachable” includes a mountable and removable state, and indicates a state where the ultraviolet irradiator 1 can be put in or taken out from the eyewear case. In the present example, the ultraviolet irradiator 1 is an irradiation kit that can be freely put in and taken out from the eyewear case C.

For example, the ultraviolet irradiator 1 is stored in the eyewear case C, and the stored ultraviolet irradiator 1 is disposed to face a bottom surface of the eyewear case C (mounting portion C1). For example, the ultraviolet irradiator 1 does not have a portion for joining to the eyewear case C. In this manner, the ultraviolet irradiator 1 can be more easily attached to and detached from the eyewear case C. However, the ultraviolet irradiator 1 may have the portion for joining to the eyewear case C. In this case, the ultraviolet irradiator 1 stored in the eyewear case C can be further stabilized.

For example, the ultraviolet irradiator 1 includes a base 2 stored in the eyewear case C so as to be freely put in and taken out from the eyewear case C, an irradiation element 3 that irradiates the eyewear E accommodated in the eyewear case C with the ultraviolet ray L, a cushion portion 4 on which a portion of the eyewear E is mounted, and a positioning portion 5 that positions the eyewear E. For example, the base 2 has a main surface 2b facing upward, and the irradiation element 3 is fixed to the main surface 2b. For example, the irradiation element 3 is an ultraviolet light emitting diode (LED). The irradiation element 3 may protrude from the main surface 2b, or may be embedded in the base 2. In a case where the irradiation element 3 is embedded in the base 2, the irradiation element 3 may be embedded in the base 2 so that a height of the irradiation element 3 with respect to the base 2 is flush with the main surface 2b.

For example, the irradiation element 3 irradiates a front surface E21 of the lens E2 of the eyewear E with the ultraviolet ray L. As an example, the irradiation element 3 has a rectangular plate shape. For example, the ultraviolet irradiator 1 includes the positioning portion 5 located in a center in the first direction D1, a pair of the irradiation elements 3 respectively disposed on one side and the other side in the first direction D1 of the positioning portion 5, and a pair of the cushion portions 4. For example, a central portion in the first direction D1 including the bridge E8 of the eyewear E is mounted on the positioning portion 5, and the rim E7 and the lens E2 are respectively located on one side and the other side in the first direction D1 when viewed from the positioning portion 5.

For example, the front surface E21 of the lens E2 is curved in a projection shape. In addition, the front surface E21 of the lens E2 is a location of the eyewear E which is easily exposed to outside air. Accordingly, the eyewear E can be efficiently sterilized by irradiating the front surface E21 with the ultraviolet ray L. For example, the ultraviolet ray L is a near ultraviolet ray. In this case, as described above, the wavelength of the ultraviolet ray L is 200 nm or longer and 380 nm or shorter.

As an example, the ultraviolet ray L is an ultraviolet C-wave (hereinafter, referred to as a UVC in some cases). The UVC is the ultraviolet ray having the wavelength of 100 nm or longer (or 200 nm or longer) and 280 nm or shorter, and shows a strong sterilizing action on the eyewear E by irradiating the eyewear E. In addition, irradiation of the ultraviolet ray L by the irradiation element 3 is performed only in a state where the eyewear case C is closed. In this manner, it is possible to prevent the ultraviolet ray L from leaking outward of the eyewear case C, and safety is ensured.

For example, the eyewear E is mounted on the base 2 in a state where a portion (for example, a lower portion) of the rim E7 is mounted on the cushion portion 4. As a specific example, the lens E2 extends obliquely upward from the portion of the rim E7 mounted on the cushion portion 4, and the lens E2 extending obliquely upward is irradiated with the ultraviolet ray L. As an example, the irradiation element 3 is disposed in a region including the center in the second direction D2 of the base 2, and the cushion portion 4 is disposed on one end side (front side) in the second direction D2 of the base 2.

For example, the cushion portion 4 is formed in a horizontally long oval shape having a pair of long sides extending in the first direction D1 and aligned in the second direction D2. In this manner, the cushion portion 4 can face a wide region of the rim E7. For example, the cushion portion 4 is made of rubber, and is formed of silicone rubber, as an example. A material of the cushion portion 4 is not limited to the rubber, and may be an elastic material or a flexible material different from the rubber. In addition, the cushion portion 4 may be omitted, and for example, instead of the cushion portion 4, the base 2 may be made of the rubber, or at least a portion of the positioning portion 5 may be made of the rubber.

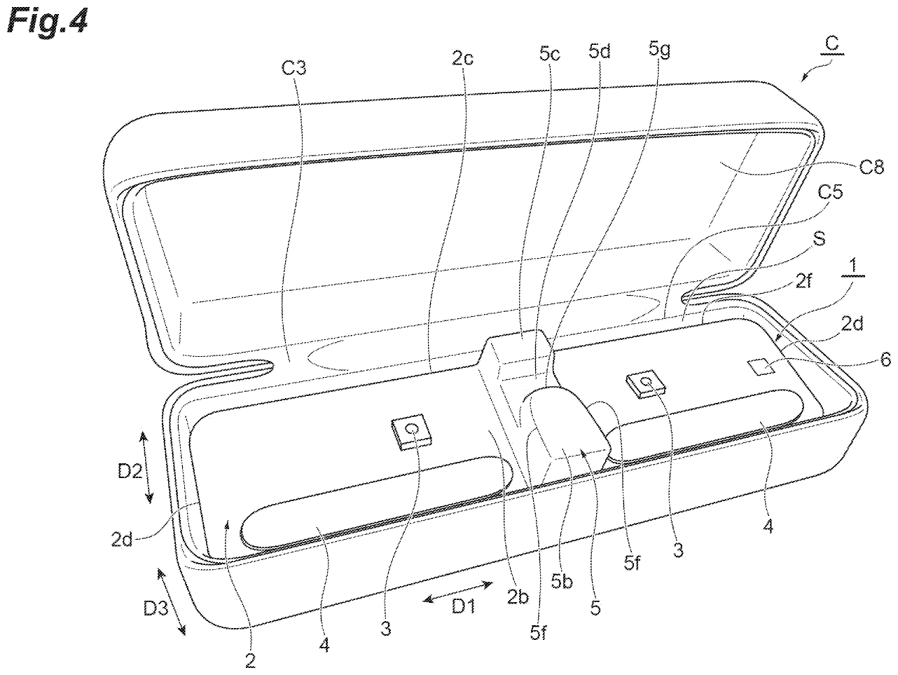

FIG. 4 is a perspective view illustrating the eyewear case C and the ultraviolet irradiator 1 in a state where the eyewear E is taken out from FIG. 2. As illustrated in FIGS. 2 and 4, for example, the base 2 has a long side 2c extending in the first direction D1 and a short side 2d extending in the second direction D2, and has a plate shape having a thickness in the third direction D3. As an example, a shape of the base 2 in a plan view is a rectangular shape having rounded corners. However, the shape of the base 2 is not particularly limited.

For example, a material of the base 2 is a resin. However, the material of the base 2 is not limited to the resin, and can be changed as appropriate. For example, in a state where the base 2 is stored in the eyewear case C, a gap S may be formed between an outer surface 2f of the base 2 and an inner surface C5 of the eyewear case C. For example, the gap S may have such a size that a finger can enter the gap S. In this case, it is possible to easily take out the base 2 from the eyewear case C. In addition, a finger hooking recess portion recessed inward of the base 2 from a portion of the outer surface 2f of the base 2 may be formed. In this case, the finger can be hooked on the recess portion, and the base 2 can be taken out from the eyewear case C. Accordingly, the base 2 can be more easily taken out.

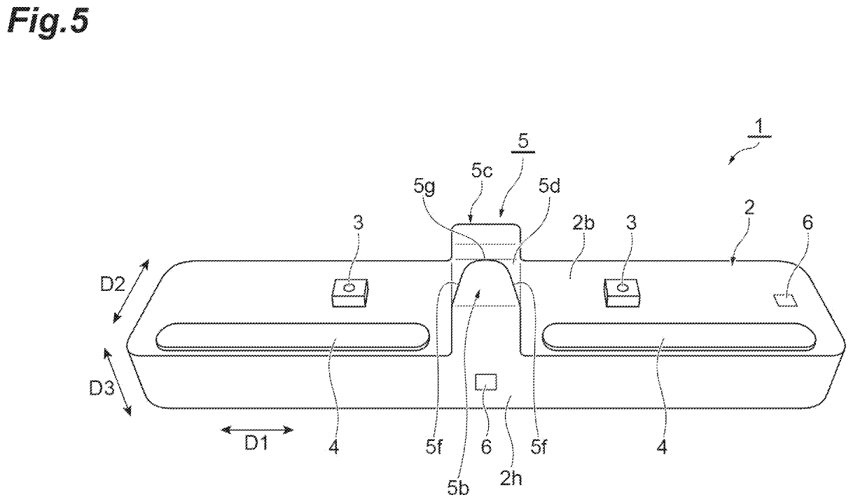

FIG. 5 is a perspective view illustrating the ultraviolet irradiator 1 taken out from the eyewear case C. As illustrated in FIGS. 2, 4, and 5, the positioning portion 5 has a projection shape protruding from the main surface 2b of the base 2. For example, the positioning portion 5 has a first projection portion 5b protruding on one side in the second direction D2, a second projection portion 5c protruding on the other side in the second direction D2, and a recess portion 5d recessed between the first projection portion 5b and the second projection portion 5c. For example, a height of the recess portion 5d is greater than a height of the main surface 2b of the base 2.

For example, the height of the first projection portion 5b with respect to the main surface 2b is greater than the height of the second projection portion 5c with respect to the main surface 2b. As an example, the base 2 is disposed in the eyewear case C so that the second projection portion 5c is located on the hinge portion C3 side. For example, the first projection portion 5b has a pair of inclined portions 5f inclined so that a width of the first projection portion 5b is narrowed toward a center side in the second direction D2, and a curved portion 5g that connects end portions of the pair of inclined portions 5f on the center side in the second direction D2 and protrudes in the second direction D2. In this way, the width of the first projection portion 5b is narrowed toward the center in the second direction D2. Accordingly, it is easy to dispose the eyewear E (for example, the pad E9).

The second projection portion 5c is provided on a side opposite to the first projection portion 5b across the recess portion 5d, and has a rectangular parallelepiped shape, for example. The second projection portion 5c is a portion which the bridge E8 of the eyewear E faces. For example, the recess portion 5d may be a portion which a portion of the rim E7 and a portion of the pad E9 enter. As a specific example, a portion on the center side in the first direction D1 of each rim E7 and a portion of each pad E9 enter the recess portion 5d. The bridge E8 is mounted on the second projection portion 5c, and a portion of the rim E7 and the pad E9 enter the recess portion 5d. In this manner, a position of the eyewear E with respect to the positioning portion 5 is determined, and a state where the lens E2 floats with respect to the irradiation element 3 is maintained. Therefore, it is possible to prevent the eyewear E from coining into contact with the irradiation element 3.

As described above, the irradiation element 3 and the cushion portion 4 are respectively disposed on one side and the other side in the first direction D1 when viewed from the positioning portion 5. Each of the cushion portions 4 linearly extends from the positioning portion 5 to the end portion side in the first direction D1. For example, each of the pair of irradiation elements 3 is provided at a position closer to the positioning portion 5 than the center of each of the cushion portions 4 extending in the first direction D1. That is, each of the irradiation elements 3 is provided on a side of the positioning portion 5 in the center of each of the cushion portions 4.

For example, the ultraviolet irradiator 1 further includes a sensor 6 that detects an open/closed state of the eyewear case C storing the ultraviolet irradiator 1. As an example, the sensor 6 is a photoelectric sensor. In this case, the sensor 6 includes an emitting unit that emits light and a light receiving unit that receives the light reflected by the emission of the light emitted from the emitting unit. The sensor 6 serving as the photoelectric sensor detects whether or not an object exists around the sensor 6, based on the amount of the light received by the light receiving unit.

The sensor 6 detects whether or not the ultraviolet irradiator 1 is stored in the eyewear case C and the eyewear case C is closed. For example, the sensor 6 serving as the photoelectric sensor emits the light from the emitting unit, and detects whether or not the inner surface (for example, the inner surface C5 or the bottom surface C8) of the eyewear case C exists around the sensor 6. In a case where the sensor 6 detects that the inner surface of the eyewear case C exists around the sensor 6, the sensor 6 detects that the eyewear case C storing the ultraviolet irradiator 1 is closed.

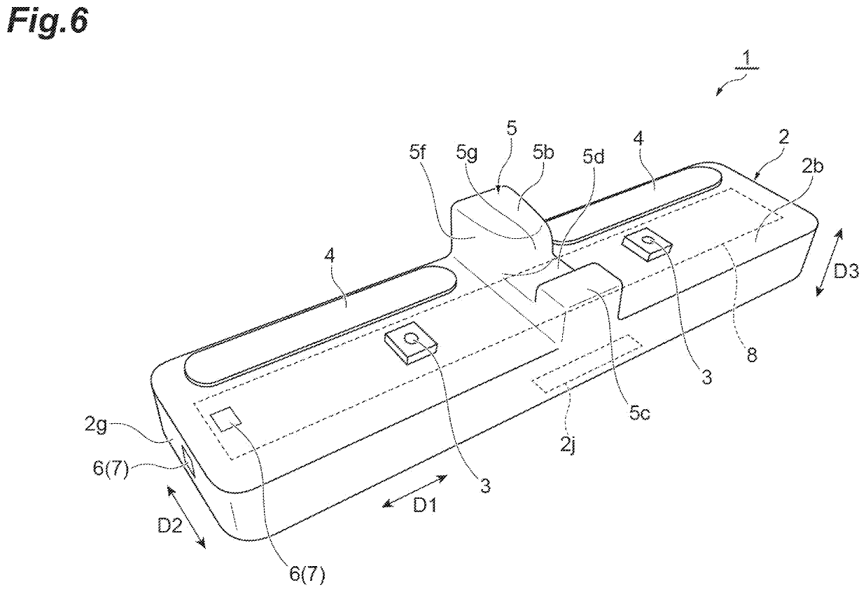

FIG. 6 is a perspective view when the ultraviolet irradiator 1 is viewed in a direction different from that in FIG. 5. As illustrated in FIGS. 5 and 6, each of the sensors 6 is fixed to each of three locations on the main surface 2b of the base 2, an end surface 2g facing the first direction D1 of the base 2, and a side surface 2h facing the second direction D2 of the base 2. Each of the sensors 6 disposed in each of the three locations detects the open/closed state of the eyewear case C. In this manner, it is possible to determine the open/closed state with higher accuracy.

In the above-described configuration, an example has been described as follows. The sensor 6 is the photoelectric sensor, and each of the sensors 6 is disposed in each of the main surface 2b, the end surface 2g, and the side surface 2h. However, a type, the number, and a disposition location of the sensor 6 are not limited to the above-described example, and can be changed as appropriate. For example, the sensor 6 may be a brightness sensor that detects ambient brightness (light amount) or a piezoelectric sensor that detects a closed state of the eyewear case C.

In addition, the number of the sensors 6 may be 2 or less or 4 or more. For example, the sensor 6 may be disposed on only one surface out of six surfaces of the base 2. Alternatively, the sensor 6 may be disposed on two surfaces, four or more surfaces, or on each of all surfaces out of the six surfaces of the base 2. As the disposition location of the sensors 6 and the number of the sensors 6 increase, the open/closed state of the eyewear case C storing the ultraviolet irradiator 1 can be detected with higher accuracy.

As an example, the base 2 includes a dry battery accommodating portion 2j for accommodating a dry battery. The dry battery accommodating portion 2j accommodates the dry battery that supplies electric power to the above-described irradiation element 3. In this way, the electric power is supplied to the ultraviolet irradiator 1 by the dry battery. Therefore, wiring extending from the base 2 can be no longer necessary, and a configuration of the ultraviolet irradiator 1 can be simplified. As an example, the dry battery accommodating portion 2j is provided on a surface opposite to the main surface 2b. Electric power supply means in the ultraviolet irradiator 1 is not limited to the above-described dry battery, and may be a rechargeable battery, for example.

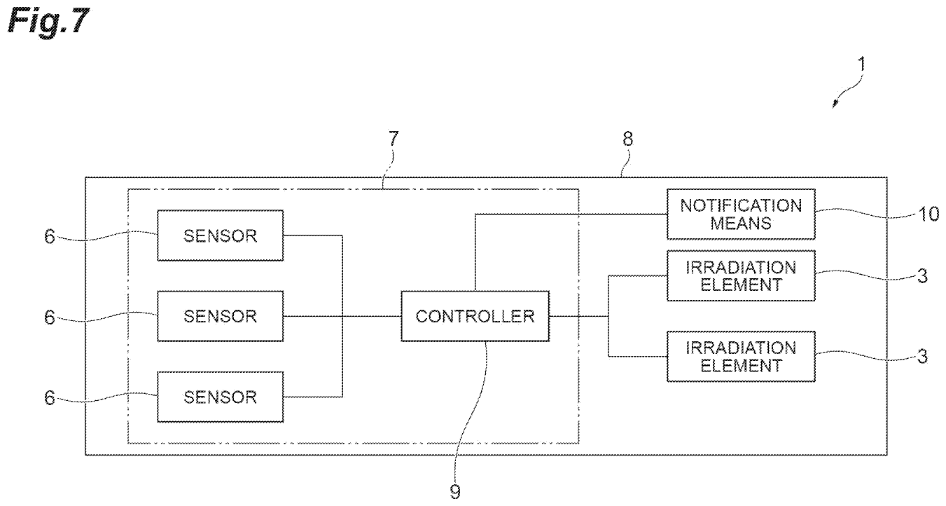

The ultraviolet irradiator 1 includes lighting means (switch) 7 for turning on the above-described irradiation element 3, and notification means (notifier) 10 for providing a notification that the lighting means 7 turns on the irradiation element 3. FIG. 7 is a block diagram illustrating a configuration of the irradiation element 3, the lighting means 7, and the notification means 10. As illustrated in FIGS. 6 and 7, for example, a circuit board 8 is provided inside the base 2.

The irradiation element 3 and the sensor 6 each are electrically connected to each other via a circuit board 8. The lighting means 7 includes the sensor 6 and the controller 9, and the controller 9 controls irradiation of the ultraviolet ray L from each of the irradiation elements 3. For example, intensity of the ultraviolet ray L used for irradiating the eyewear E by the irradiation element 3 is constant, and may be determined in advance by an experiment or a simulation. In addition, the intensity of the ultraviolet ray L may be variable, and adjusting means capable of adjusting the intensity of the ultraviolet ray L may be provided. Alternatively, the controller 9 may automatically control the intensity of the ultraviolet ray L.

For example, the notification means 10 is connected to the controller 9, and receives control of the controller 9 to notify a user of the ultraviolet irradiator 1 whether or not the irradiation element 3 is turned on. For example, the notification means 10 may be a sound output unit for outputting sound when the controller 9 turns on the irradiation element 3, or may be a vibrator vibrated when the controller 9 turns on the irradiation element 3. In addition, the notification means 10 may be communication means for notifying an information terminal (for example, a mobile terminal such as a smartphone or a tablet) of the user of the ultraviolet irradiator 1 that the irradiation element 3 is turned on when the controller 9 turns on the irradiation element 3.

In this case, for example, the notification means 10 notifies the mobile terminal of the user that the irradiation element 3 is turned on via e-mail or an application. In this way, the notification means 10 notifies the user that the irradiation element 3 is turned on. Accordingly, the user of the ultraviolet irradiator 1 can recognize that the irradiation element 3 is turned on inside the eyewear case C. The notification means 10 can prevent the user from erroneously opening the eyewear case C while the eyewear case C is irradiated with the ultraviolet ray L. Furthermore, through the notification means 10, the user can recognize a turned-on state (whether or not the irradiation element 3 is turned on) of the irradiation element 3, which cannot be directly recognized in a state where the eyewear case C is closed, whether the battery is dead, and whether there is a problem in that the irradiation element 3 is not turned on. Furthermore, as in the notification means 10, the ultraviolet irradiator 1 may include remaining amount notification means for notifying a remaining amount of the above-described dry battery or the battery.

Each of the plurality of sensors 6 detects whether the ultraviolet irradiator 1 is stored in the eyewear case C and whether the eyewear case C is closed. For example, the controller 9 turns on the irradiation element 3 when all of the sensors 6 detect the closed state of the eyewear case C. For example, the controller 9 may turn on the irradiation elements 3 for a fixed period of time (as an example, 10 seconds or more and 10 minutes or less), and may turn off each of the irradiation elements 3 after the fixed period of time elapses. In this case, the eyewear E can be automatically irradiated with the ultraviolet ray L for the fixed period of time. Accordingly, it is possible to adopt a configuration without requiring a manual switch.

A time for the irradiation element 3 to irradiate the eyewear E with the ultraviolet ray L may be constant as described above, or may be determined in advance by an experiment or a simulation, as in the intensity of the ultraviolet ray L. In addition, a time for irradiating the eyewear E with the ultraviolet ray L may be variable, or adjusting means capable of adjusting an irradiation time (turned-on time) of the ultraviolet ray L may be provided. Alternatively, the controller 9 may automatically control the irradiation time of the ultraviolet ray L.

Next, an operational effect obtained from the ultraviolet irradiator 1 according to the present example will be described in detail. As illustrated in FIGS. 2 and 3 as an example, in the ultraviolet irradiator 1, the base 2 is stored in the eyewear case C, the irradiation element 3 for irradiating the eyewear E with the ultraviolet ray L is fixed to the base 2, and the irradiation element 3 is turned on by the lighting means 7. The base 2 to which the irradiation element 3 for irradiating the eyewear E with the ultraviolet ray L is fixed is attachable to and detachable from the eyewear case C.

Therefore, the base 2 and the irradiation element 3 can be taken out from the eyewear case C, and the base 2 and the irradiation element 3 which are taken out can be stored in another eyewear case. Therefore, the base 2 provided with the irradiation element 3 can be stored in the ready-made eyewear case. Accordingly, an irradiation function can be diverted to the ready-made eyewear case. Since the irradiation function can be diverted to the ready-made eyewear case, it is not necessary to purchase a new eyewear case in a case where the irradiation function of ultraviolet ray L is to be used.

Therefore, it is economical since the ready-made eyewear case can be continuously used. In addition, the irradiation element 3 and the base 2 are provided separately from the eyewear case C. Therefore, electric wiring or an electric circuit is not incorporated in the eyewear case C, and the circuit board 8 can be integrated in the base 2. As a result, each configuration of the ultraviolet irradiator 1 and the eyewear case C can be simplified.

The base 2 may include a positioning portion 5 that positions the eyewear E with respect to the base 2. In this case, the eyewear E accommodated in the eyewear case C is positioned by the positioning portion 5. Accordingly, the eyewear E can be stably disposed inside the eyewear case C. Therefore, the stably disposed eyewear E can be irradiated with the ultraviolet ray L from the irradiation element 3. Accordingly, the eyewear E can be more efficiently irradiated with the ultraviolet ray L.

The base 2 may have the main surface 2b to which the irradiation element 3 is fixed, and the positioning portion 5 may have a projection shape protruding from the main surface 2b. In this case, the positioning portion 5 has the projection shape protruding to the main surface 2b of the base 2 to which the irradiation element 3 is fixed. Therefore, when the eyewear E is positioned by the positioning portion 5 having the projection shape, it is possible to adopt a configuration in which the eyewear E is less likely to come into contact with the irradiation element 3.

The irradiation element 3 may irradiate the eyewear E with the UVC as the ultraviolet ray L. In this case, the UVC which is particularly effective in sterilization can be used for irradiating the eyewear E. Accordingly, the eyewear E can be more efficiently sterilized by irradiating the eyewear E with the UV-C.

As illustrated in FIGS. 6 and 7, the lighting means 7 may have the sensor 6 that detects an open/closed state of the eyewear case C in a state where the base 2 is stored in the eyewear case C, and the controller 9 that controls the irradiation of the irradiation element 3 in accordance with the open/closed state detected by the sensor 6. The controller 9 may turn on the irradiation element 3 when the closed state of the eyewear case C is detected. In this case, the sensor 6 detects the open/closed state of the eyewear case C storing the base 2, and the controller 9 turns on the irradiation element 3 when the closed state of the eyewear case C is detected. Therefore, it is possible to adopt a configuration in which the irradiation element 3 is not turned on when the eyewear case C storing the base 2 is not closed. Accordingly, safety can be improved by preventing unnecessary irradiation of the irradiation element 3.

The sensor 6 may be a photoelectric sensor that converts light into an electric signal. In this case, unlike a push button, no external force is required. Accordingly, a configuration of the sensor 6 itself can be simplified. In addition, since the photoelectric sensor is used, the irradiation element 3 is not turned on, when the base 2 is not stored in the eyewear case C, or when the eyewear case C is open and there is no object around the base 2.

The base 2 may have a plate shape, the lighting means 7 may have the plurality of sensors 6, and the plurality of sensors 6 may be attached to each of the plurality of surfaces of the base 2. In this case, each of the plurality of sensors 6 is attached to each of the plurality of surfaces of the base 2. Even in a case where one sensor 6 performs erroneous detection, the other sensor 6 performs proper detection. Accordingly, an erroneous operation of the lighting means 7 can be more reliably prevented. Therefore, it is possible to control the irradiation element 3 to be turned on with higher accuracy.

As illustrated in FIG. 3, the ultraviolet irradiator 1 may include the plurality of irradiation elements 3, and the plurality of irradiation elements 3 may be disposed at positions facing a forward-facing surface (for example, the front surface E21) of the lens E2 of the eyewear E. The forward-facing surface of the lens E2 of the eyewear E is a portion exposed forward when viewed from a wearer wearing the eyewear E, and is a portion easily exposed outward in respective portions of the eyewear E. Therefore, the plurality of irradiation elements 3 face the forward-facing surface of the eyewear E to which bacteria are likely to adhere. Accordingly, the bacteria can be more effectively sterilized by irradiating the surface with the ultraviolet ray L from each of the irradiation elements 3.

As illustrated in FIG. 7, the ultraviolet irradiator 1 may include the notification means 10 for providing a notification that the lighting means 7 turns on the irradiation element 3. In this case, the notification means 10 notifies the user whether or not the eyewear E is irradiated with the ultraviolet ray L. Accordingly, it is possible to easily recognize whether or not the eyewear E is irradiated with the ultraviolet ray L.

As illustrated in FIG. 2, the base 2 may include the cushion portion 4 with which a portion of the eyewear E comes into contact. In this case, the eyewear E is accommodated in the eyewear case C in a state where a portion of the eyewear E is in contact with the cushion portion 4. Therefore, even in a case where an impact is applied to the eyewear case C, the cushion portion 4 cushions the impact. Accordingly, the eyewear E can be protected.

It is to be understood that not all aspects, advantages and features described herein may necessarily be achieved by, or included in, any one particular example embodiment. Indeed, having described and illustrated various examples herein, it should be apparent that other examples may be modified in arrangement and detail.

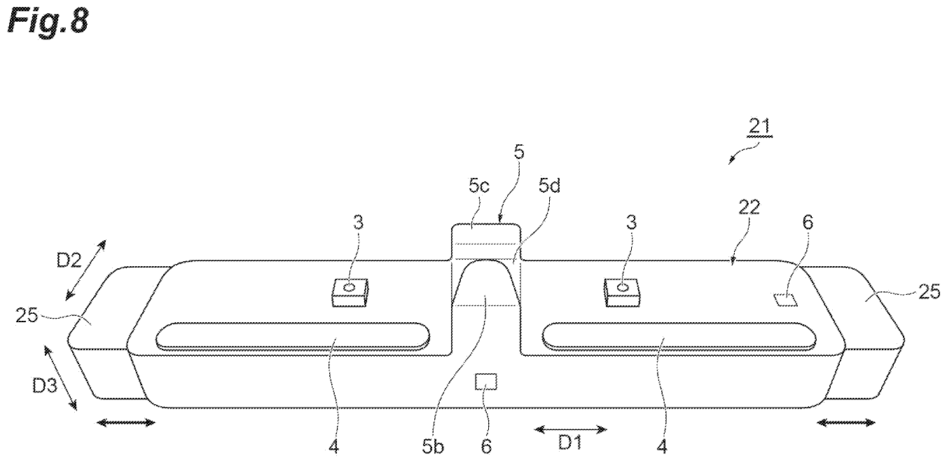

For example, in the above-described example, the ultraviolet irradiator 1 including the base 2 having the plate shape has been described. However, the shape of the base is not limited to the plate shape. As illustrated in FIG. 8, the ultraviolet irradiator 21 may have a variable size. The ultraviolet irradiator 21 according to a modification example includes a base 22 and a slide portion 25 having a nested shape with respect to the base 22. The slide portion 25 can be freely put into the base 22 and taken out from the base 22. The slide portion 25 can be freely put into the base 22 and taken out from the base 22 at an end portion of the base 22 in the first direction D1, and a protruding amount of the slide portion 25 protruding from the base 22 is adjusted. In this manner, it is possible to adjust the length of the ultraviolet irradiator 21 in the first direction D1.

FIG. 8 illustrates an example in which the ultraviolet irradiator 21 includes a pair of the slide portions 25 aligned along the first direction D1. In a case of the ultraviolet irradiator 21, the protruding amount of the slide portions 25 with respect to the base 22 can be adjusted on each of both sides in the first direction D1. However, the ultraviolet irradiator 21 may include the slide portion 25 in one end in the first direction D1, and the protruding amount of the slide portion 25 with respect to the base 22 may be adjustable on only one side (one of the right side and the left side) in the first direction D1. As described above, in the ultraviolet irradiator 21, the length in the first direction D1 can be adjusted. Accordingly, the ultraviolet irradiator 21 can be exactly stored in the eyewear cases having different lengths in the first direction D1. That is, the length in the first direction D1 of the ultraviolet irradiator 21 can be variable depending on a size of the eyewear case.

In the above-described example, the ultraviolet irradiator 21 including the slide portion 25 that can be freely put into the base 22 and taken out from the base 22 at the end portion of the base 22 in the first direction D1 has been described. However, for example, instead of the slide portion 25, the ultraviolet irradiator 21 may include a slide portion that can be freely put into and taken out in the second direction D2 with respect to the base 22, or a slide portion that can be freely put into and taken out in the third direction D3 with respect to the base 22. In this case, the ultraviolet irradiator can be exactly stored in the eyewear case having a different length in the second direction D2 or the eyewear case having a different length in the third direction D3.

In the above-described example, an example has been described in which the sensor 6 serving as the photoelectric sensor detects the closed state of the eyewear case C to automatically turn on the irradiation element 3. As described above, the sensors that detect the closed state of the eyewear case C may be any sensor other than the photoelectric sensor, or may be a combination of the photoelectric sensor and another sensor. As an example, a photoelectric sensor and a piezoelectric sensor may be provided as the sensors. In addition, the lighting means for turning on the irradiation element 3 may be any means other than the sensor, and may be a manual operation switch, for example.

In the above-described example, the ultraviolet irradiator 1 including the base 2 having the plate shape and made of the resin has been described. However, a material of the base 2 is not particularly limited. For example, at least a portion of the main surface 2b of the base 2 may include a mirror-finished surface. In this case, the ultraviolet ray L emitted for irradiation from the irradiation element 3 is reflected by the mirror-finished surface. Accordingly, it is possible to further improve efficiency in irradiating the eyewear E with the ultraviolet ray L.

In the above-described example, the ultraviolet irradiator 1 including the pair of irradiation elements 3 aligned along the first direction D1 has been described. However, the number and the disposition aspect of the irradiation elements 3 are not limited to the example of the ultraviolet irradiator 1. For example, one irradiation element may be disposed in a region including the center in the first direction D1, or an irradiation element may be further provided at a position facing each of the pads E9 in addition to the pair of irradiation elements 3.

In the above-described example, the ultraviolet irradiator 1 provided with the positioning portion 5 having the projection shape protruding from the main surface 2b of the base 2 has been described. However, a shape of the positioning portion is not limited to the projection shape protruding from the main surface 2b of the base 2. The shape of the positioning portion may be any shape that can fix a portion of the eyewear. For example, a recess portion may be provided on the main surface 2b of the base 2, and the irradiation element 3 may be disposed on a bottom surface of the recess portion. In this case, the positioning portion that does not protrude from the main surface 2b can be provided. As a specific example, a recess portion may be formed on the main surface 2b, and the eyewear may be fitted into the recess portion. In this manner, the positioning portion for positioning the eyewear may be provided. In addition, the positioning portion can be omitted. For example, instead of the positioning portion, a cushion portion or a cloth-like portion interposed between the eyewear E and the eyewear case C may be provided. As a specific example, when the eyewear E is disposed in the cushion portion having the recess portion disposed in a state where the irradiation element 3 does not protrude inside the eyewear case C, the eyewear E can be protected without adopting the positioning portion. In addition, the cushion portion or the cloth-like portion may be provided between the eyewear E and the base 2 (main surface 2b). In this case, the cushion portion or the cloth-like portion may have a hole portion exposed in an aspect in which the irradiation element 3 does not protrude. In this manner, the eyewear E can be prevented from coining into contact with the irradiation element 3, and the eyewear E can be protected by the cushion portion or the cloth-like portion.

We claim all modifications and variations coining within the spirit and scope of the subject matter claimed herein.

1. An ultraviolet irradiator that irradiates an eyewear with an ultraviolet ray, comprising:
a base configured to be stored in an eyewear case that accommodates the eyewear;
an irradiator fixed to the base, and irradiating the eyewear accommodated in the eyewear case with the ultraviolet ray; and
a switch configured to turn on the irradiator,
wherein the base is attachable to the eyewear case and detachable from the eyewear case.

2. The ultraviolet irradiator according to claim 1,
wherein the ultraviolet irradiator is an irradiation kit that is a separate component from the eyewear case, and can be mounted in the eyewear case and can be removed from the eyewear case.

3. The ultraviolet irradiator according to claim 1,
wherein the base includes a positioning portion that positions the eyewear with respect to the base.

4. The ultraviolet irradiator according to claim 3,
wherein the base has a main surface to which the irradiator is fixed, and
wherein the positioning portion has a projection shape protruding from the main surface.

5. The ultraviolet irradiator according to claim 4,
wherein the positioning portion comprises:
a first projection portion protruding on one end of the base;
a second projection portion protruding on the other end of the base; and
a recess portion recessed between the first projection portion and the second projection portion.

6. The ultraviolet irradiator according to claim 5,
wherein a height of the recess portion is greater than a height of the main surface of the base.

7. The ultraviolet irradiator according to claim 5,
wherein a height of the first projection portion with respect to the main surface is greater than a height of the second projection portion with respect to the main surface.

8. The ultraviolet irradiator according to claim 5,
wherein the first projection portion has a pair of inclined portions so that a width of the first projection portion is narrowed toward a center of the base.

9. The ultraviolet irradiator according to claim 8,
wherein the first projection portion has a curved portion that connects two end portions of the inclined portions at a side of the center of the base and protrudes toward the second projection portion.

10. The ultraviolet irradiator according to claim 1,
wherein the irradiator irradiates the eyewear with an ultraviolet C-wave.

11. The ultraviolet irradiator according to claim 1,
wherein the switch comprises:
a sensor that detects an open/closed state of the eyewear case in a state where the base is stored in the eyewear case; and
a controller that controls irradiation of the irradiator in accordance with the open/closed state detected by the sensor, and
wherein the controller turns on the irradiator when the sensor detects a closed state of the eyewear case.

12. The ultraviolet irradiator according to claim 11,
wherein the sensor is a photoelectric sensor that converts a light into an electric signal.

13. The ultraviolet irradiator according to claim 11,
wherein the base has a plate shape,
wherein the switch comprises a plurality of sensors, and
wherein the plurality of sensors are attached to each of a plurality of surfaces of the base.

14. The ultraviolet irradiator according to claim 13,
wherein the switch comprises three sensors, each of the three sensors are attached to each of three surfaces of the base, the three surfaces intersect each other, and the three surfaces include a main surface to which the irradiator is fixed.

15. The ultraviolet irradiator according to claim 1, further comprising:
a plurality of irradiators,
wherein the plurality of irradiators are disposed at positions facing a forward-facing surface of a lens of the eyewear.

16. The ultraviolet irradiator according to claim 1, further comprising:
a notifier configured to provide a notification that the switch turns on the irradiator.

17. The ultraviolet irradiator according to claim 16,
wherein the notifier provides the notification to an external information terminal.

18. The ultraviolet irradiator according to claim 1,
wherein the base comprises a cushion portion with which a portion of the eyewear comes into contact.

19. The ultraviolet irradiator according to claim 1,
wherein the base has a main surface to which the irradiator is fixed and at least a portion of the main surface comprises a mirror-finished surface.

20. The ultraviolet irradiator according to claim 1,
wherein the irradiator is embedded in the base, and
wherein a height of the irradiator with respect to the base is flush with the main surface.
